(19) United States
(12) Patent Application Publication
(10) Pub. No.: US 2021/0153776 A1
(43) Pub. Date: May 27, 2021

(54) METHOD AND DEVICE FOR SIZING AN INTERATRIAL APERTURE

(71) Applicant: InterShunt Technologies, Inc., St. Louis, MO (US)

(72) Inventors: Chris Minar, Excelsior, MN (US); Gil M. Vardi, Town and country, MO (US); John M. Berns, Eagan, MN (US)

(21) Appl. No.: 17/105,427

(22) Filed: Nov. 25, 2020

Related U.S. Application Data
(60) Provisional application No. 62/940,194, filed on Nov. 25, 2019.

(51) Int. Cl. A61B 5/107; A61B 5/0215; A61B 5/00; A61B 5/11

(52) U.S. Cl. CPC A61B 5/1076; A61B 5/0215; A61B 5/7264; A61B 5/11; A61B 5/6898

(57) ABSTRACT

The invention relates to a method, software, and device 300 used to determine a size or size range 260 for an aperture in the interatrial septum of a heart. The invention relates to software 200 with a learning model 310 which helps a clinician select a surgical shunt size for a patient, monitoring the patient post operatively, and for making shunt modification recommendations, based on specific and aggregate patient data.

### BACKGROUND OF THE INVENTION

### Field of the Invention

The invention relates to medical devices and methods of medical treatment. The invention relates to a method, software, and device used to determine a size or size range for an aperture in the interatrial septum of a heart. The invention relates to software with a learning model which helps a clinician select a surgical shunt size for a patient, monitoring the patient post operatively, and for making shunt modification recommendations, based on specific and aggregate patient data.

### Background Art

There are some medical conditions that are treated by creating an opening between body chambers in order to create a connection between the chambers. The heart has an interatrial septum or wall that separates the left atrium and the right atrium. In certain heart failure patients (e.g., heart failure with preserved ejection fraction (diastolic dysfunction)) there is a need to allow blood flow from the left atrium to the right atrium to reduce left atrial pressure. Shunt or apertures in the tissue may be formed by removing tissue, dilating the tissue and fixating it, or be placing a scaffold to hold it open. Likewise, certain other cardiovascular diseases and conditions, such as congenital heart diseases and pulmonary hypertension may be treated by making a similar shunt.

It is important to not make shunts such as an interatrial shunt too large, for both safety and effectiveness reasons. As an example, in patients with oversized interatrial shunts the right side of the heart can get too much shunted blood volume, increasing pressure and causing right sided heart failure. The same oversized shunt may also reduce the atrial pressure gradient causing blood to potentially flow from right to left. This right to left flow causes a risk of stroke. Systemic cardiac output may also be reduced by interatrial shunt sizes that are too big. The upper end of the shunt size range is defined by many patient variables, including patient size, atrial pressures, expected cardiac remodeling, and many more.

On the lower end of shunt sizes (0-3 mm) the benefit in symptom relief may not be worth the risk of the shunt creation surgical procedure. It is also believed that these smaller shunt sizes may close over time. Any tearing or cutting through the fossa ovalis endothelial surface will be followed by an angiogenic healing response from the local smooth muscle cell and fibroblast derived factors. Both of these factors depend on patient specific variables.

Therefore, it would be desirable to have a system that is capable of identifying a shunt size or shunt size range recommendation based on historical and patient specific data.

### BRIEF SUMMARY OF THE INVENTION

The present invention solves these needs by providing a wearable sensor device for recording dynamic data for shunt prescription calculation that includes a blood pressure monitor, a heart monitor that includes a sensor, an interface for receiving data from a user, a motion sensor, a signal processor for processing a data set from the blood pressure monitor, the heart monitor, the interface, and the motion sensor. The signal processor configured to record the data set along with meta data identifying the data set. The device further includes a storage medium configured to store the data set and a communications module configured to communicate the data set to a server, the server comprising a server communications module and a shunt prescription software.

In some embodiments the communications module is a smartphone. The device may further include an oxygenation sensor. The device may further include an ambient data sensor. The device may further include an exertion sensor.

In other embodiments the invention is a method of treating heart failure. The method includes receiving, via a communications module, a data set comprising data from a wearable sensor device. The data includes data from a blood pressure monitor, data from a heart monitor, data from a motion sensor, and meta data. The data set is input into a machine learning algorithm trained to identify a shunt prescription. Based on that prescription, an interatrial shunt is created according the shunt prescription.

In some embodiments the method further includes the step of curating the data. In some embodiments the machine learning algorithm is a deep neural network algorithm. In other embodiments the shunt prescription is a range of values. The shunt prescription can include a target value and a lower value. In other embodiments the method further includes the step of measuring the success of the interatrial shunt creation. In some embodiments the method further includes inputting an indicator of success of the interatrial shunt creation into the machine learning algorithm.

In another embodiment the invention includes a method of treating heart failure that includes the steps of (1) receiving a data set comprising patient data, the data including data from a blood pressure monitor, data from a heart monitor, data from a motion sensor, and meta data; (2) removing unnecessary data from the data set; (3) curating the data into a standardized format; (4) inputting the data set into a machine learning algorithm trained to identify a shunt prescription; and (5) surgically creating an interatrial shunt according the shunt prescription.

In embodiments the method may further include the step of curating the data. In some embodiments the machine learning algorithm is a deep neural network algorithm. In other embodiments the shunt prescription is a range of values. In embodiments the shunt prescription includes a target value and a lower value. The method may further include the step of measuring the success of the interatrial shunt creation. The method may further include inputting an indicator of success of the interatrial shunt creation into the machine learning algorithm.

### BRIEF DESCRIPTION OF THE DRAWINGS

FIG. 1 is a schematic view of a wearable data collection device and system according to the present disclosure;

FIG. 2 is a flow chart of example workflow for providing a shunt prescription;

FIG. 3 is a system configuration for processing, coordinating, and directing the shunt sizing system;

FIG. 4 is a a flow chart of example workflow for training and validating a machine learning system according to the present invention;

FIG. 5a is a flow chart of the exemplary data flow of medical data to be used in the present invention;

FIG. 5b is a flow chart of the exemplary data flow of medical data to be used in the present invention;

### DETAILED DESCRIPTION OF THE PREFERRED EMBODIMENTS

In general, the invention comprises a method, software, and device used to determine a size or size range for an aperture in the interatrial septum of a heart. The invention relates to software with a learning model which helps a clinician select a surgical shunt size for a patient, monitoring the patient post operatively, and for making shunt modification recommendations, based on specific and aggregate patient data, as well as a device for monitoring patient specific data.

Shunts which are believed to be effective, safe and remain patent are believed to be between 3-10 mm in diameter; however this range may need to be broadened depending on a range of variables. Kaye, in Effects of an interatrial shunt on rest and exercise hemodynamics: Results of a computer simulation in heart failure predict that shunt sizes between 8-9 mm would achieve most of the shunt benefit for heart failure patients, while keeping flow safely left to right.

However, this model is on a small set of heart failure patient data. There are several factors which may impact the need to broaden this shunt size range, move it up or down the scale, change over time, and be specific to patient specific data. Considering the potential range of diseases which may be treated with interatrial shunting, stage within the disease, various comorbidities, range of patient sizes, variability in expected shunt discharge coefficients, and the variation in expected shunt healing, it is reasonable that an atrial shunt circular diameter of between at least 0-15 mm may be necessary.

Creating permanent apertures through the interatrial septum, and in particular, the fossa ovalis, have been enabled through various medical device technologies. One procedure uses a balloon to create a hole in the septum, others cut and remove tissue, some place tubes or scaffold like stents to hold the tissue open, and yet others dilate and fixate the tissue open. Prescribing the optimal shunt size is not an easy endeavor because if the shunt is too small it may heal closed or not provide sufficient clinical therapy to justify the shunt creation procedure, and, if the shunt is too large the excess blood shunting may cause blood to flow in the wrong direction, or cause too much blood to flow in the correct direction, both causing a multitude of potential health issues, like stroke and right side heart failure. The invention prescribes an optimized interatrial shunt size for specific patient needs, prescribing a shunt large enough to not close and provide enough therapy, while at the same time limiting the risks of an oversized shunt.

Flow through the shunt is exponentially related to the diameter of the shunt, so small changes in shunt diameter equate to a large change in the amount of flow. Proper shunt sizing should also take into account patient specific data or physiological variables which make prescribing an optimal shunt size difficult.

As noted above, certain medical conditions such as heart failure, pulmonary artery hypertension, congenital heart disease, and similar conditions, are treated by creating an opening between body chambers in order to create a connection between the chambers. In certain heart failure patients (e.g., heart failure with preserved ejection fraction (diastolic dysfunction)) there is a need to allow blood flow from the left atrium to the right atrium to reduce left atrial pressure. Likewise, certain other heart diseases and conditions, such as congenital heart diseases and pulmonary hypertension may be treated by making an interatrial opening; however, the goal is to create a right-to-left shunt to reduce the high right-sided pressure. The invention may allow a user to select to solve or specify a shunt size, or shunt size range, of a primary selected disease, such as heart failure with preserved ejection fraction. The invention may also allow the user to input from very many or very few data inputs and ask the invention to solve a shunt size output, or range of shunt size outputs, for all possible diseases. In general, the more data types and the more data points that are entered, the more accurate the algorithm will be accounting for the various comorbidities any patient may have.

Disclosed herein are a series of medical devices and software to identify a shunt size or a shunt size range for a patient. The shunt size is ideally adjusted to the specific anatomy, physiology, and diagnosis of the patient. Thus, the diagnosis may be an input data piece both for training and validating the software, as well as for providing a specific shunt prescription. Initially, a physician will seek to diagnose the patient by conducting a series of tests as disclosed in U.S. patent application Ser. No. 16/677,455, filed Nov. 7, 2019, and incorporated herein by reference, as well as the patient specific testing discussed herein. In order to better understand the patient's unique physiology and needs, the physician may also prescribe the use of a portable or wearable data collection device.

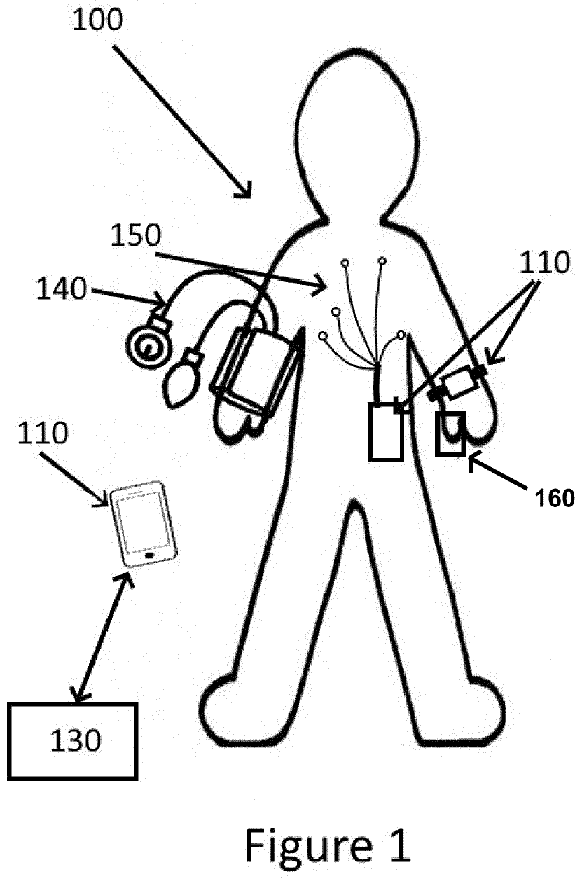

With reference to FIG. 1, a wearable data collection device 110 is worn or carried by the patient 100. It collects dynamic individual patient data 120. Wearable data collection device 110 may be wrist worn, e.g., a smart watch, carried, e.g., a smart phone, or more be a medical specific wearable akin to a Holter monitor. It may also be a combination of modules, e.g., a smart watch or Holter monitor that utilizes the smart phone for processing or communication with the shunt prescription system and software 130, which ideally uses both the individual patient data 120 and cumulative or historical patient data 250.

Wearable data collection device 110 may seek to collect key data that is used to diagnose heart failure. For example, wearable data collection device 110 may collect blood pressure data, ECG data, oxygenation data. To collect this data, the wearable data collection device may include a blood pressure monitoring apparatus 140. For example, the system may include a wireless blood pressure cuff, and the patient 100 may be prompted to use or remove the wireless blood pressure cuff periodically, or use it permanently for a period of diagnosis. Likewise, the system may include a heart monitor 150. The heart monitor 150 may be a series of electrodes that may monitor ECG data. Alternatively the data collection device may include optical heart rate monitors 150 which may optically penetrate the skin to detect blood flow. The system may include a pulse oximeter 160. Other data collection devices 110 may require the patient 100 to place a finger over a sensor on the wearable data collection device, and may prompt the patient 100 to do so periodically. For example, a data collection device 110 itself may include a pulse oximeter for measuring shortness of breath.

Because key diagnosis data includes shortness of breath, ECG data and blood pressure data an ideal wearable data collection device 110 would include the ability to measure each of these elements. Technology may enable the ability to take other key data dynamically, such as lung sound, BNP levels (see herein), PCWP (Pulmonary Capillary Wedge Pressure), PCAP (Pulmonary Capillary Pressure), PA pressure (Pulmonary artery pressure), RA pressure, and cardiac output. To the extent this data may be taken by a wearable device, they would ideally be included and the data collected. In addition, previously acquired data for each of these may be stored on the wearable device or associated cell phone 110 for use in calculations.

Ideally the wearable data collection device 110 also collects data regarding exertion. For example, the wearable data collection device may collect a distance traveled, a number of steps, elevation changes. Each data item would preferably include a time stamp, and as the system can link a particular set of readings, for example a particular blood pressure, ECG reading, and exertion reading together with a time. As discussed above, the data gathered by the wearable data collection device 110 is communicated through a smart phone, bluetooth, LAN, radio, Wi-Fi, or other data link to the shunt prescription system and software 130, discussed in detail below. The wearable data collection device 110 may determine particular times where a particular measurement is desirable, and may prompt the user or the appropriate module to take that measurement when needed. Likewise, in the event real time data transmission is possible between the shunt prescription system and software 130 and the wearable data collection device 110, the shunt prescription system and software 130 may determine that additional data is desirable and direct the wearable data collection device 110 to prompt the user or the corresponding module to gather that data.

While the wearable data collection device 110 may not be capable of gathering all of the data relevant to a heart failure diagnosis, the physician, user, or shunt prescription system and software 130 may provide additional static data regarding the patient 100 to the data collection device. In addition, the user may input additional data on the wearable data collection device 110, such as if the user weighed himself in the morning, noticed a medical issue such as shortness of breath or pain, took a temperature, or the like. The wearable data collection device 110 may also collect ambient data, such as temperature, humidity, smog, location, or other ambient information.

Data collection device 110 may be used to continuously collect dynamically changing data from the user regarding the user's heart health. For example, data collection device 110 may collect data regarding user 100's heart rate, heart rate variability, pulse pressure wave morphology, and other features that may provide predictive value regarding the heart health of the user, and be used as patient input data before shunt creation or after. Data collection device 110 may also provide feedback, alerts, reminders, and other indications to patient 100 that may encourage self-care and tell the patient and clinician if the shunt size needs to be adjusted up, down, or closed. The sizing system may include a variety of data collection, data entry, data processing and data output equipment, such as body weight scales, ECG devices, implanted blood pressure sensors, blood pressure cuffs, exercise equipment, digital stethoscopes, and other devices (not shown) that provide an indication of one or more aspects of the health of patient 100. The output of data collection device 110 may be transferred to wearable computing device, wrist worn dynamic sensor systems, additional computing devices, shunt prescription system and software 130 and/or cloud computing devices for analysis, including use in a predictive machine learning model for collecting static data, dynamic data, and pooled patient data, as discussed below.

The shunt prescription system and software 130 may allow for a clinician or data technician to input and view all patient static and dynamic data, run various scenarios, create adjustments to the machine learning algorithm (discussed below) prior to calculating optimal shunt size, shunt size recommended range, and risks. The clinician may then use this data to prescribe surgical procedures. Patient specific post procedure data collection can be used to update the quality of the patient pool data, which in turn may be used to update the software based machine learning model or algorithm for specifying general future patient shunt size, or shunt size adjustments. The shunt sizing system may also be used to trend data such that intervention can be adopted prior to an irrecoverable medical situation. The wearable data collection device 110 may include an algorithm such as a machine learning algorithm to identify when immediate intervention is warranted, and then utilize a communication means (discussed herein) to alert the user or a physician.

In some cases, the shunt prescription system and software 130 may identify patients that should be excluded from a shunt prescription. For example, the software may identify that the patient has a lower pressure gradient between left and right atria, and at that lower pressure gradient a shunt is not beneficial. The software may also identify a situation where the pressure gradients may result in a reversal of shunt direction due to elevated right atrial pressure at particular points, or during exercise. For most diagnosis, e.g., HFpEF, the LA pressure should be significantly higher than RA pressure in the patient to be suitable for shunting. Preferably the LA pressure should be higher at both rest and during exertion. However, this is patient, diagnosis, and comorbidity specific, and as such the system 130 is trained to identify how much higher for a specific patient with a specific diagnosis. Likewise, in some patients the relationship between changes in PCWP and left ventricle cardiac output might indicate a shunt is preferred or is not preferred. Thus, the system is also trained to identify the relationship that best indicates a shunt.

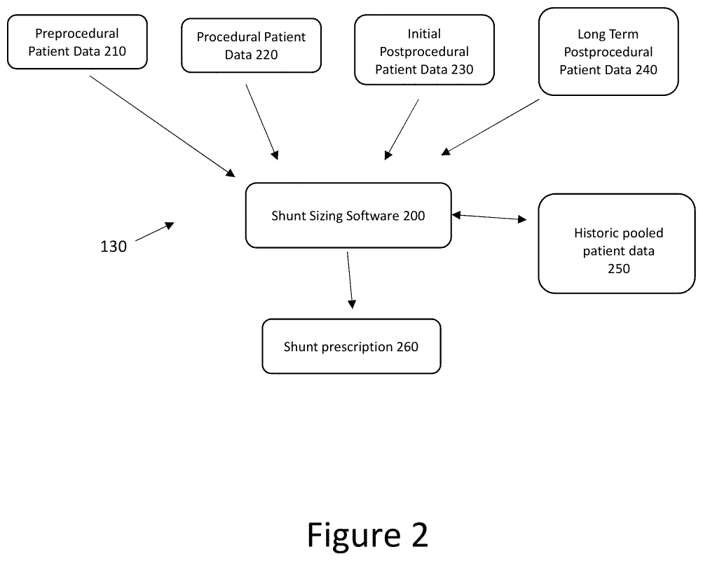

With reference to FIG. 2, shunt prescription system 130 and software 200 includes several inputs and outputs. Patient pre-procedural data 210, patient procedural data 220, initial patient post procedural data 230 and long term patient post procedural data 240, both static and dynamic, as well as historic pooled patient data 250 may all be inputs to define a shunt prescription 260 for heart failure patients. The historic data 250 thus includes data gathered at different time points for each patient. It includes some, most or preferably all of the input data types that would be included with data 210-240. While the data gathered pre-procedurally 210 would have a different time point than the post procedural data 230 and 240, it is also true that even with the pre-procedural data there may be multiple measurements at multiple times, even of the same test, e.g., multiple pulse measurements, multiple EKG tests, and the like.

The patient data inputs 210, 220, 230, 240, 250 may include those described earlier, and may also include some or all of the data which was used to diagnose the heart failure and additional metrics, including, but not limited to, general laboratory data, electrocardiography data, Kansas City Cardiomyopathy Questionnaire (KCCQ) scores (physical limitation score, symptom stability score, symptom frequency score, symptom burden score, total symptom score, self-efficacy score, quality of life score, social limitation score, overall summary score, and clinical summary score), HF hospitalizations, level of pulmonary congestion, a basic measurement of vital signs, electrocardiogram (ECG), an echocardiography and other imaging modalities to assess cardiac output, ventricular contraction and filling, atrial size, and cardiac valve function, etc., measurement of pulmonary capillary wedge pressure (PCWP), body weight, 6 minutes' walk test, body frame size, BMI, ECG data, demographic information, medications taken before, during, and after hospitalization, patient vital signs, patient lab results, patient weight change during hospitalization, echocardiogram results, comorbidities, blood pressure, blood tests to check for chemicals such as brain natriuretic peptide (BNP and Nterminal pro-B-type natriuretic peptide (NT-proBNP), a stress test, maximum systolic slope, systolic rise time, election time, dicrotic notch height, dicrotic notch timing, total pulse pressure, pulse arrival time, pulse transit time, heart rate, heart rate variability, pressure wave morphology, activity level, step count, sleep time, sleep quality, posture, reflected wave arrival time, cardiac catheterization and/or an MRI or CT scan. In addition, transthoracic echocardiography (TTE) or transeophogeal echocardiography (TEE) may be used to confirm the absence of any current holes between the chambers of the heart, and if there is an existing shunt a quantitative assessment of size and flow is determined.

The patient data may also include patient anatomical size, neurohormonal effects, drugs, stage of disease, chamber compliance, complicating comorbidities, tissue/shunt thickness, previous or anticipated shunt size reduction due to healing, and post procedure physiological remodeling. Other patient data may include amount of lung congestion, Creatinine (a chemical waste product of creatine, an amino acid, excreted in urine), Blood urea nitrogen (a waste product produced as a result of digestion of protein; an indicator of kidney function), Hemoglobin (a protein responsible for transporting oxygen in blood), White blood cell count, Platelets (a type of blood cell that helps form clots to stop bleeding), patient happiness score, Albumin (a liver-produced protein that helps keep fluid in the bloodstream and not leak into other tissues), Red blood cell distribution. Due to the complexity and number of these patient data input variables which need to go into calculating proper shunt size, and the interdependencies between them, an intelligent system is preferred to tailor shunt size for specific patients.

In one embodiment, the patient data inputs may also include all comorbidities which may potentially impact the prescription, either through direct impact, like hemodynamic flow reduction due to PAD, or indirect impact from variables which might impact remodeling. Finally, patient morbidity and causes of mortality may also be inputs.

All of these input variables may be later also used as output variables. That is, in an advanced embodiment the software may predict how the variables may change after shunt creation, as the patient ages, and the body remodels. In addition, in one embodiment the physician may input target values for one or more variables, and the software may provide options, e.g., shunt size and or medication, for achieving those values. The system may also identify additional information that would be valuable for the calculation in the instant case, as well as variables that are suspect and should be re-measured.

The system may also provide information on the type of shunt that would be ideal, e.g., a bare shunt, a circular shunt, an oval shunt, a series of slits cut (such as an x-pattern), multiple oval shunts, and the like as described in U.S. Pat. No. 9,814,483, U.S. patent application Ser. No. 15/900,127, filed Feb. 20, 2018, or U.S. patent application Ser. No. 16/677,455, filed Nov. 7, 2019, each of which are entirely incorporated herein. The system may also recommend the use of an implant, drug treatment around the shunt, or another method of modifying the size or morphology of the shunt, both for present results as well as long term results, including the shunt shrinking due to healing.

The shunt prescription 260 output may include a binary output indicating if a patient may benefit from an interatrial shunt, what the possible risks are and their risk levels, the ideal interatrial shunt size, minimum and maximum shunt sizes, and as noted above, the software may predict how all or many of the input variables may change after a shunt is created and the heart remodels. After the initial shunt creation further patient data input into the system can be used to monitor trends, correct recommendation on the shunt size, assess risk levels, and adjust predicted outcomes. Collectively these outputs, before, during and after a shunt creation procedure may be included in a shunt prescription 260. The patient may have a means to collect and send input data to the shunt sizing software in real time, and be able to get direct output in real time. Alternatively the output alarms could be set such the patient is not notified unless a certain alarm parameter is met.

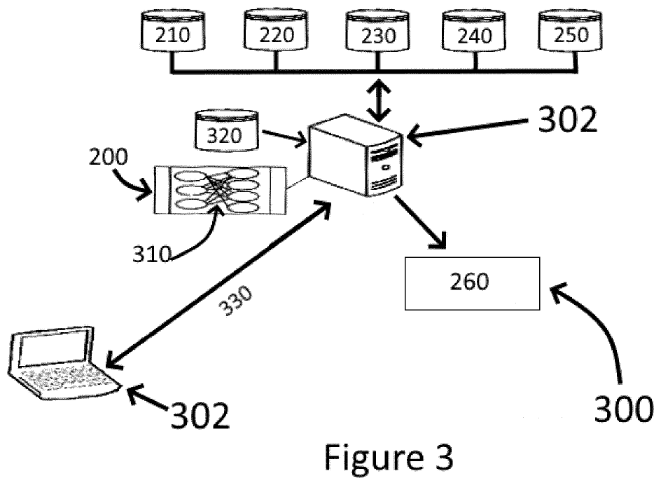

As shown in FIG. 3, the machine learning system 300 may include one or more computers or servers 302 for data processing, storage, data retrieval, data input, and the like. In some implementations, medical data, such as data 210, 220, 230, 240, 250 can be processed by a compiled binary or software executed with the system 300's processor and the memory of the server 302, to provide a shunt prescription 260. The binary or other software executed with the server 302 may implement one or more machine learning models on the medical data, as discussed below. Typically, when the output is a desired shunt size or shunt range, e.g., shunt prescription 260, the model will use a supervised learning algorithm 310 such as a neural network, deep neural network, support vector machine model, decision tree, random forest algorithm, extreme gradient boosting, or the k-nearest neighbor algorithm. With use of the learning model 310, the machine learning system 300 is able to produce accurate and detailed shunt prescription 260 through a data-driven cycle of training, processing, and expert verification of results.

Using a prepared historical data set 250, the algorithm 310 is trained to provide a shunt prescription 260. In some examples, the trained detection algorithm 310 may process historical pooled data inputs 250 and one or more of current data inputs 210, 220, 230, 240 at different levels of the model. Alternatively, an unsupervised algorithm such as Apriori or K-means may be utilized to identify unknown or unappreciated factors in shunt sizing.

In one embodiment, the machine learning system 300 produces data indications to identify future diagnostic or interventional efforts that would affect the shunt prescription or efficacy. In some implementations, the machine learning system 30 may establish descriptors, markings, annotations, or additional metadata for data 210, 220,230, 240,250, may indicate the presence of particular comorbidities or conditions, the absence of certain identified conditions, the likelihood/probability/or other certainty score of such identified conditions, and other related outputs from the operation of a recognition algorithm on the medical data 210, 220, 230, 240, 250.

Prior to the machine learning system 300 receiving the data, the data should be prepared for use in the system. In particular, data gathered from many different devices (such as numerous different types of ECG machines), will need to be standardized, or may be limited to particular sources. Alternatively, the data can be entered with an indication of the source for the algorithm to differentiate between different data sources. The more detailed the metadata for a particular datum, the broader the applicability of the developed model. However, it will be more difficult to train the model. Accordingly, while a large historical data set may be preferred, a more homogenous data set enables quicker training. At present the data set for actual interatrial shunt procedures is very limited. Thus, in one embedment the invention uses a real time model of the cardiovascular system to generate data to train the machine learning system 300. This data model is then phased out as actual patient data is generated.

When the system 300 receives data it will process the data for further handling in the workflow. This processing may include converting the data to a different format, standardizing the data, or rejecting the data. The machine learning system 300 may also operate to extract metadata from each data file. For example, the extracted metadata may include header data providing patient information and medical facility information for the facility that gathered the data. The machine learning system 300 may then store all or part of the extracted information in a study record that may be correlated with other like data.

As shown in FIG. 3, a machine learning model 310 within the shunt sizing software 200 and learning model 310 may be initially trained by all or a subset 320 of historic pooled patient data 250. After the data is input into the machine learning model 310 the output can be compared to expected or known outputs in a model calibration routine 330, which feeds weight and bias adjustments back into the machine learning model 310, via back propagation, regression or similar. A model performance assessment may be done on a variety of machine learning models, such as random forest, gradient boosting, support vector machine, logic regression, logic regression with lasso.

Then the model which seems the most accurate for the given set of data and variables may be selected for utilization. Data samples from the pool may be initially be sorted into critical and non-critical, based on which input variable ranges have the greatest impact on the output variable ranges. As an example, patient frame size is likely to be a significant predictor of necessary 1L shunt size. So it is optimal to sort and use training data which covers the broadest possible range of patient frame sizes. Also, it will be important that the training data include both those patients who have had optimal results from their shunt creation in the form of lower heart classification, improved KCCQ scores, lower hospitalization rates, etc. The training data should also include data from those patients who have had the worst follow-up results, such that the model is trained to not prescribe shunt size prescriptions which will potentially give poor results. The data samples used for model training include all variables, and ideally the full range of critical variable data.

Applicants have identified ECG results, echocardiograms, stress test, blood tests (as discussed herein), x-ray, cardiac output, atrial size, and patient frame as critical variables for shunt sizing. In particular, the software is designed to focus on the PCWP and RA pressures both before and after a procedure. Thus, the shunt size prescription should take into account the pre operative PCWP and RA pressure, as well as the targeted post operative PCWP and RA pressure. As a result, the data used should include this information, and it should be more heavily weighted in the software.

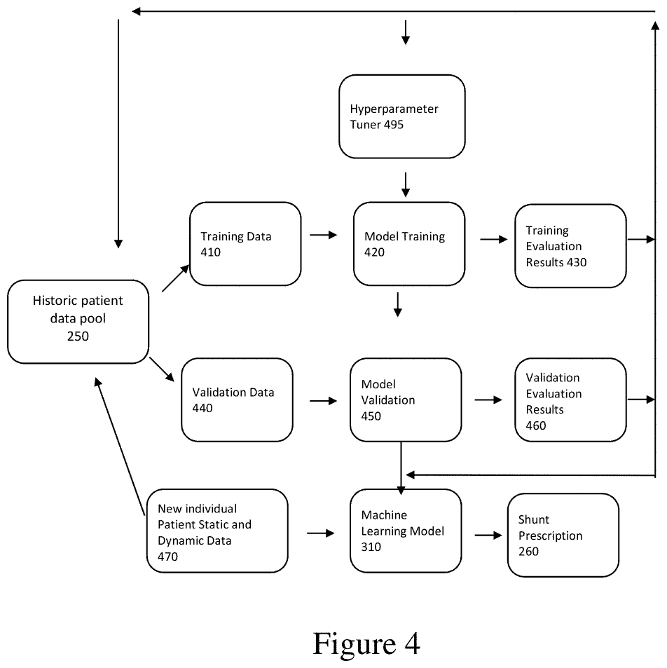

FIG. 4 shows a more detailed model for training, validating, commercial implementation for continuously updating a machine learning model 310 within the software 200. A training data subset 410 is pulled from the historical patient pooled data set 250. In one embodiment the training data is 75% of the pooled data set 250. In another embodiment it is 60%. After the data is curated, training data 410 is fed into the model for model training 420, resulting in training evaluation results 430. In particular, the critical variables identified above are given an increased weight in the model. These results may be run through multiple models or algorithms 310, depending on the results. The model 310 may be adjusted based on the training evaluation results 430. Likewise, the data may be tuned, added too, or reduced based on the results 430.

These training evaluation results 430 are typically fed into a hyperparameter tuner 495 to adjust the model, in a preferred embodiment using Bayesian optimization. However, if the results match measured results then they may also be fed back into the patient data pool 320 as calculated results. Similarly a validation data subset 440 of historical patient pooled data set 250 is used for model validation 450. In one embodiment the validation data is 25% of the pooled data set 250. In another embodiment there are two validation cohorts of 20% each. In a similar manner to training data 410, the validation evaluation results 460 may be fed into either the hyperparameter tuner 495 or the pooled patient data 320 or both. Once validated, new patient data 470 may then be fed into the shunt sizing software 200 and the machine learning model 310. The software 200 and/or model 310 calculate a shunt prescription 260. The shunt prescription 260 can be fed into the hyperparameter tuner 495 to continue model training, and into the historic patient data pool 250.

In this way the software helps the machine learning model to continuously learn. FIG. 4 shows how a model may continue to learn from each individual patient dataset even after initial calibration from pooled data sets, by use of continued back propagation routines, to adjust weighting and bias. In particular, the critical variables identified above are given an increased weight in the model. Likewise, data generated outside the model and the specific patients can be pooled and fed into the model 310.

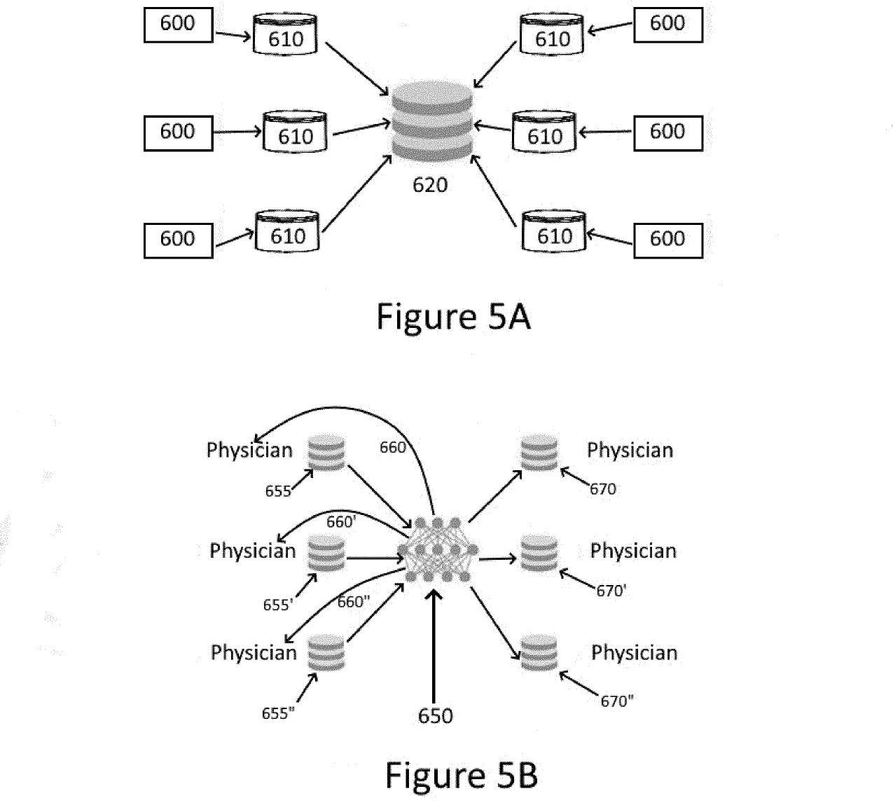

Initially the model 310 is trained by historic pooled patient input and output data 250, simulated patient data, or both. The accuracy of model 310's output—shunt prescription 260 or an identification of unknown or unappreciated factors in shunt sizing—will depend greatly on the quantity and quality of the historical pooled data 250. Data preparation includes several steps. First off, all applicable regulations, privacy rules, and ethical rules must be observed before the data may be used. Often the data must be cleaned of certain identifying information. The data must then be reviewed for quality control, curated, structured, and expertly labeled, as well as consistently labeled. With reference to FIG. 5A, a medical provider 600 will gather relevant local patient data 610. Local patient data may be in a standardized format, such as an electronic health record (EHR). Likewise, the system may require that data be in a standardized EHR format. Local data 610 is then provided to a central data storage 620. Data technicians may ensure that the data is properly labeled, curated, and complies with all regulations either prior to transmittal from provider 600, or at the centralized data storage facility. Alternatively, the data can be cleaned during model development, or the data 610 may be partially cleaned and/or curated locally and partially at a centralized setting.

Once the data has been properly cleaned by removing or modifying data that is incorrect, incomplete, irrelevant, duplicated, or improperly formatted it will be utilized in training the model. Likewise, during training the data technician may employ dimensionality reduction to remove data nodes that are redundant to other data, or to combine nodes. In an optional step the data technician will seek to create visualizations, tests, or relationship matrices for the data and its relationship to shunt sizing. Expertly identifying the key data components as discussed above and their relationship to shunt sizing will assist in model training.

A first set of new patient data in the form of static and dynamic pre-procedural data 210 is received for a user, fed into the machine learning software 200, and one or more shunt prescriptions 260 are generated by a machine-learning model based on the set of data. The initial shunt sizes may include the ideal shunt size, a range of sizes, and shunts on the low end of an ideal range, knowing that the shunt can be increased in size during follow-up procedures more easily than reducing the shunt size.

Likewise, the software may recommend a particular type of shunt. For example, the software may recommend a circular shunt, oval shunt, shunts formed by slits, shunts formed by implants, post shunt formation treatment (tissue treatment to prevent healing and shunt closure), and the like, as discussed above.

Clinical or cloud computing is used in aggregate, subset, single institutional form, or single clinician form to train the machine learning model 310. In aggregate a single patient pool of data 320 is collected and shared between clinical sites, which allow for a broader data set to more properly adjust the algorithm via machine learning. However, there are situations where a clinician or clinic may only want data from a single institution, individual clinician, or individual patient. In addition, the system may favor data from a particular physician or institution in providing a result for further procedures from that physician, institution, or another location. Accordingly, in data training the system 300 allows the data technician to give greater weight to data from a particular source in training the system, or to train the system 300 using data from a particular source.

The amount of data utilized for the training set may determine which learning algorithm or model 310 is utilized. For a large amount of data, a deep neural net training model is preferred by the inventors. Due to the large number of input characteristics, or features, the neural network has a large number of input neurons. As a result, the neural network may also have a very large number of hidden layers, each with its own parameters, before passing to the output layer. As information passes through the neurons, or nodes it is weighted by expert input and model training as to the key information identified above. During training the data scientist will use back propagation, feeding the prediction errors back through the network modifying the weights of each neural connection until the error level is minimized. However, a deep neural network can overfit the data 13) due to the excessive number of layers, and as such some of the historical pool data must be not be used for training, and can only be used for validation to relax the overfit. That is, the first time the model sees this validation data 440 is in validation. At present the inventors' preferred deep leaning technique is performed on a very powerful computer enhanced with GPU(s) rather than the handheld's disclosed herein due to the computer power requirements of a deep neural network. However, a handheld may be preferred for calculating a shunt size prescription and post operative shunt monitoring to identify complications.

In the event that the system 300 is to be employed solely within one clinic, or for one physician, the limited data subset gives preference to a support vector machine model 310. If the model is to be formed quickly for a new patient, the support vector machine model may be faster to train, and may be more readily trained in a limited computing or time environment. Generally a supervised learning model like a supervised neural net or a SVM requires data with accurate labels for training.

A machine-learning training pool patient data set for calibrating the machine learning model within the software, within the system may be split into D and S data sets which respectively represent historic pooled dynamic data 500 and static data 510. The pooled data is initially input into a single classifier or regressor for shunt size calculation. The classifier sorts for optimized follow up data, based on initial data and shunt size prescription. To make sure that the machine learning model is not to tightly fit to an unwanted subset the feature selection and regularization ideally include pooled data sets from multiple institutions, multiple physicians and multiple patient types (various comorbidities).

The static and dynamic pooled training data is fed into a single classifier or regressor in the model, to calculate patient suitability for a shunt and a shunt size prescription. The dynamic and static data 500, 510 can also be fed into separate classifiers. Also, dynamic data 500 can be used to update static data 510 and then be fed into a single static classifier. Classifiers may be adjusted based on dynamic data, medication or other variables. The data can combine features or keep them separate. For example, similar comorbidities might be combined. Also, separating data for initial training may help to calibrate the model more efficiently with less data. The best set of input variable features and weights can be determined manually by the clinician or by the machine learning model 310.

Due to the large number of both dynamic and static input variables, it is ideal that a deep neural net training model be used, though other training models may also work as well or better. A first model is trained by the historic data and has nodes in a penultimate layer which are trained to be predictive of the known shunt sizes which gave the best related patient outcomes from the shunt. The model only needs to be trained once with the historical data, then later training can be done patient by patient as follow-up output data is available, or the new output data can be added to the historical patient pool and the model retrained on a periodic basis or once a specified number of new patients have been added to the pool.

After a patient receives a shunt the software can preferentially weight new data from the patient's new inputs to adjust its outputs to more closely align with the patient's specific details. The software may be set to increase input variable weighting for each new input. In this way the model may be trained to learn a specific patient or subset of patients. Multilevel models may be used, such that once the classifiers and models are trained patient specific preprocedural data is entered into an intermediate model. The intermediate output values from the intermediate model are then concatenated with the dynamic procedural variables as available to form the feature vector for the final model and shunt size prescription. Later, in a similar way another concatenation may be done with post procedural patient data such that a new shunt prescription could include a new shunt size adjustment or other medical prescriptions. Deep neural net training models 310 may also be used. In this way heart and whole body remodeling may be accounted for.

That is to say one intermediate or final model layer may be to account for remodeling, or remodeling may be accounted for in a single main model, or a completely separate model may be used for post shunt patients. For specific patients, the program or software may be configured in a way which the machine-learning model is taught based on the patient's static data only, or combined with historical data from a large patient data pool. This data may then be used with future dynamic patient specific data to update the model on an ongoing basis. Specific classifiers may be used on new patient data to determine shunt size and other prescriptions. Training classifiers and models with only patient specific data may be useful in limiting the impact the multitude of important variables certain to be left out of the model. However, patient specific classifier and model training can only occur after the initial shunt is created and post procedure data collected. Even then, variables such as right ventricular remodeling can only be trained by leveraging data from a larger patient population data pool. So it may be useful to combine multiple models to maximize the value of both specific patient data and pooled patient data. Right ventricular remodeling can be measured via measuring the size of the right ventricle on echocardiograms, detecting a “flattening of the interventricular septum” on echocardiogram, RA pressure, and PA pressure. Accordingly, in those embodiments that seek to address remodeling, these factors will be paramount.

Training dynamic data classifiers may include dampener algorithms to account for medications typically given to heart failure patients (e.g., rate limiters, diuretics, vasodilators) which have a significant acute effect that can be observed within dynamic data. Also for specific patients a machine learning model similar to the one used for determining the shunt size prescription may also be used to track the patients progress post shunt creation procedure, from seconds to decades after the shunt is created. The model may prescribe opening the shunt more, closing it partly or all the way, or non-shunt prescriptions.

Once the model 310 is trained, the model must be evaluated. Validation data 440—data that is previously unseen by the model—is fed into the model for model validation 450. Expert medical assistance with create a series of metrics to measure the objective performance of the model, does the shunt size prescription 260 match effective, successful shunts, and avoid in effective or harmful shunts? Accordingly, in curating the training data it is critical that markers be included as to the effectiveness of the shunt. Such markers include the interatrial pressure gradient, pulmonary capillary wedge pressure (PCWP), pulmonary artery pressure (PAP), 6 minute walk test, and quality of life improvements. As a result, in this embodiment, training and validation data is utilized with heavy weighting on these factors. In particular, the software is designed to focus on the PCWP and RA pressures both before and after a procedure. Thus, the shunt size prescription should take into account the pre operative PCWP and RA pressure, as well as the targeted post operative PCWP and RA pressure. The physician may provide a preferred post operative result, or the system may identify preferred results for a given patient. For example, the authors have identified that a Qp/Qs ration of 1.3/1 would significantly reduce left atrial pressure. Thus, they system may target a Qp/Qs ration of 1.3/1, a ratio between 1.2/1 to 1.4/1 depending on the patient. A successful model must be sensitive, specific, and accurate.

After either in the initial training or the validation, the parameters 495 are tuned. Thus, the number of training steps, the learning rate, and the initialization values, and the distribution can be adjusted to improve the model. This is an iterative process.

As shown in FIG. 5B, once the model is developed, trained, tested, and tuned, it is ready to provide shunt prescription 260. There are multiple ways the shunt prescription 260 may be provided to a physician. First, the model may be developed centrally as central model 650. In usage a physician may transmit individual patient data 655, 655′ 655″ (consisting of part of or all of data 210, 220, 230, and 240) to the centralized model 650, where it is processed and providing individual shunt prescription 660, 660, 660″ back to the respective physician. Alternatively, a base model may be developed, which is then provided to local server 670, 670′, or 670″. The base model is then tuned to the needs of the local facility or physician, per the methods discussed herein. At the local facility the individual patient data 655 is run in the locally tuned model on the local server 670, to result in the shunt prescription 660. Of course, the model 650 may also be individually tuned at a central location for each local facility or physician as well. (not shown).

Dynamic data collection may be via wearable physiological monitoring devices, computers, phones, tablets, catheters, voice interface devices, implantable devices, etc., with sensors such as microphones, visible-light sensors, ultraviolet sensors temperature sensors, pressure transducing device, electrodes, a contact sensor module—EKG measuring cardiac pressure sensors, and adjust an active shunting system real time, motion sensors, accelerometer, gyroscope, and magnetometer, GPS, electromagnetic guidance systems. All of these are used to measure the patient's blood-oxygen level, pulse, blood glucose levels, or other biometric markers with optical signatures, ECG, augmentation index, maximum systolic slope, systolic rise time, ejection time, dicrotic notch height, dicrotic notch timing, total pulse pressure, reflected wave arrival time, activity level, step count, sleep time, sleep quality, posture, etc.

The machine learning model may be in in any program in any computing device(s) such as desk top or laptop computer, a tablet, a phone, a watch, in the cloud, or on an institutional server. There may be a plurality of models running in parallel or series. The patient pool data may be on a computing device or in the cloud, in RAM, optical memory (e.g., CD, DVD, HD-DVD, Blu-Ray Disc, etc.), semiconductor memory (e.g., RAM, EPROM, EEPROM, etc.), and/or magnetic memory (e.g., hard-disk drive, floppy-disk drive, tape drive, MRAM, etc.). Information may be displayed on monitors with light emitting diode (LED) array, a liquid-crystal display (LCD) array, (LCOS) array) may, active-matrix organic lightemitting diode (AMOLED) displays or quantum dot displays. Data may be transferred with two-way Bluetooth, cellular, near-field communication and/or other radios. In some implementations, communication suite may include an additional transceiver for optical (e.g., infrared) communication.

1. A wearable sensor device for recording dynamic data for shunt prescription calculation comprising:
a blood pressure monitor;
a heart monitor, the heart monitor comprising a sensor;
an interface for receiving data from a user;
a motion sensor;
a signal processor for processing a data set from the blood pressure monitor, the heart monitor, the interface, and the motion sensor; the signal processor configured to record the data set along with meta data identifying the data set;
a storage medium configured to store the data set;
a communications module configured to communicate the data set to a server, the server comprising a server communications module and a shunt prescription software.

2. The wearable sensor device of claim 1, wherein the communications module is a smartphone.

3. The wearable sensor device of claim 1, further comprising an oxygenation sensor.

4. The wearable sensor device of claim 1, further comprising an ambient data sensor.

5. The wearable sensor device of claim 1, further comprising an exertion sensor.

6. A method of treating heart failure comprising:
receiving, via a communications module, a data set comprising data from a wearable sensor device, the data comprising:
data from a blood pressure monitor;
data from a heart monitor;
data from a motion sensor; and
meta data;
inputting the data set into a machine learning algorithm trained to identify a shunt prescription;
surgically creating an interatrial shunt according the shunt prescription.

7. The method of claim 6, further comprising the step of curating the data.

8. The method of claim 7, wherein the machine learning algorithm is a deep neural network algorithm.

9. The method of claim 8, wherein the shunt prescription is a range of values.

10. The method of claim 8, wherein the shunt prescription includes a target value and a lower value.

11. The method of claim 7, further comprising the step of measuring the success of the interatrial shunt creation.

12. The method of claim 11, further comprising inputting an indicator of success of the interatrial shunt creation into the machine learning algorithm.

13. A method of treating heart failure comprising:
receiving a data set comprising patient data, the data comprising:
data from a blood pressure monitor;
data from a heart monitor;
data from a motion sensor; and
meta data;
removing unnecessary data from the data set;
curating the data into a standardized format;
inputting the data set into a machine learning algorithm trained to identify a shunt prescription;
surgically creating an interatrial shunt according the shunt prescription.

14. The method of claim 13, further comprising the step of curating the data.

15. The method of claim 13, wherein the machine learning algorithm is a deep neural network algorithm.

16. The method of claim 15, wherein the shunt prescription is a range of values.

17. The method of claim 15, wherein the shunt prescription includes a target value and a lower value.

18. The method of claim 13, further comprising the step of measuring the success of the interatrial shunt creation.

19. The method of claim 18, further comprising inputting an indicator of success of the interatrial shunt creation into the machine learning algorithm.
